(19) United States
(12) Patent Application Publication
(10) Pub. No.: US 2005/0040044 A1
(43) Pub. Date: Feb. 24, 2005

(54) System of handling dielectric particles, particularly biological cells, by means of dielectrophoresis

(76) Inventors: Marie Frenea, Thurins (FR); Bruno Le Pioufle, Brevillet (FR); Herve Lhermite, Cesson-Sevigne (FR)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/493,004

(22) PCT Filed: Oct. 21, 2002
(86) PCT No.: PCT/FR02/03595

(30) Foreign Application Priority Data
Oct. 22, 2001 (FR) 01/13600

(51) Int. Cl. G01L 1/20; C07K 1/26

(52) U.S. Cl. 204/643; 204/450

(57) ABSTRACT

A system for using dielectrophoresis to manipulate dielectric particles, in particular biological cells in suspension in a medium and subjected to the action of an alternating electric field of distribution that is made to be non-uniform by means of a regular array of electrodes suitable for defining local zones where the electric field is at a minimum in rows to concentrate particles in said local zones by the action of negative dielectrophoresis forces, wherein the array of electrodes is formed on the surface of a multilayer substrate, and wherein the same-polarity electrodes of the array are connected to respective common power supply pads via two arrays of conductor tracks which are formed at an intermediate level situated beneath the array of electrodes.

The invention relates to a system for using dielectrophoresis to manipulate dielectric particles, in particular biological cells.

### BACKGROUND OF THE INVENTION

In general, dielectrophoresis, as discovered in 1951 by Pohl, designates the force exerted by a non-uniform alternating electric field on a particle that is polarizable, but not necessarily provided with an electric charge.

One of the major applications of dielectrophoresis relates to separating particles in suspension in a medium. If a particle is more polarizable than the medium in which it is suspended, then the dielectrophoresis force will be positive and the particle will be directed towards a region in which the local electric field is at a maximum, and under opposite circumstances, the particle will be directed to a region in which the local electric field is at a minimum. In general, electric field distribution depends on the shape of the electrodes, and the magnitude of the dielectrophoresis force varies with frequency as a function of the dielectric properties of the medium and of the particles.

### OBJECTS AND SUMMARY OF THE INVENTION

An object of the invention is to devise a system having high density or a high degree of integration in order to be able to manipulate a large number of particles, which implies a special design for the disposition of the electrodes and the way in which they are powered.

To this end, the invention provides a system for using dielectrophoresis to manipulate dielectric particles, in particular biological cells in suspension in a medium and subjected to the action of an alternating electric field of distribution that is made to be non-uniform by means of a regular array of electrodes suitable for defining local zones where the electric field is at a minimum to concentrate particles in said local zones by the action of negative dielectrophoresis forces, wherein the array of electrodes is formed on the surface of a multilayer substrate, and wherein the same-polarity electrodes of the array are connected to respective common power supply pads via two arrays of conductor tracks which are formed at an intermediate level situated beneath the array of electrodes.

In an embodiment, the multilayer substrate comprises at least one base substrate, a conductor layer being deposited on the base substrate to form the two arrays of conductor tracks, and an insulating layer deposited on the conductor layer for forming the array of electrodes, the array of electrodes being connected to the arrays of conductor tracks via holes passing through the insulating layer.

In an embodiment, the electrodes are regularly spaced apart in a plurality of rows parallel to an axis X, the electrodes of any one row having the same polarity, the electrodes of two adjacent rows having opposite polarities, and the local particle-concentration zones are regularly spaced apart in a plurality of rows parallel to the axis X.

In general, the system also comprises a chamber formed above the substrate for receiving particles in suspension, the chamber being defined, for example, by a sealing gasket which surrounds at least the array of electrodes, and by a plate fitted onto the gasket, together with an alternating voltage source for feeding the two connection pads of the electrodes.

By way of example, the multilayer substrate may support an electrode array that is suitable for defining a number of local zones of the order of 1000 to 50,000 for a substrate having a side of one centimeter.

### BRIEF DESCRIPTION OF THE DRAWINGS

Other advantages, characteristics, and details of the invention appear from the following explanatory description made with reference to the accompanying drawings given purely by way of example, and in which:

FIG. 1 is a diagrammatic plan view of an example of an electrode array formed on the surface of a top insulating layer of a multilayer substrate, and suitable for use in manipulating dielectric particles by means of dielectrophoresis;

FIG. 2 is a plan view of two arrays of conductor tracks feeding the array of electrodes in FIG. 1;

FIG. 3 is a section view on line III-III of FIG. 2 to show the positions of the arrays of conductor tracks shown in FIG. 2 relative to the array of electrodes shown in FIG. 1;

FIGS. 4 and 5 are diagrams showing two other possible embodiments for the array of electrodes shown in FIG. 1;

FIGS. 6a and 6b show two other shapes for electrodes; and

FIG. 7 is a diagrammatic view of an embodiment of a system for using dielectrophoresis to manipulate dielectric particles in suspension in a medium in contact with the electrode array of FIGS. 1, 4, or 5.

### MORE DETAILED DESCRIPTION

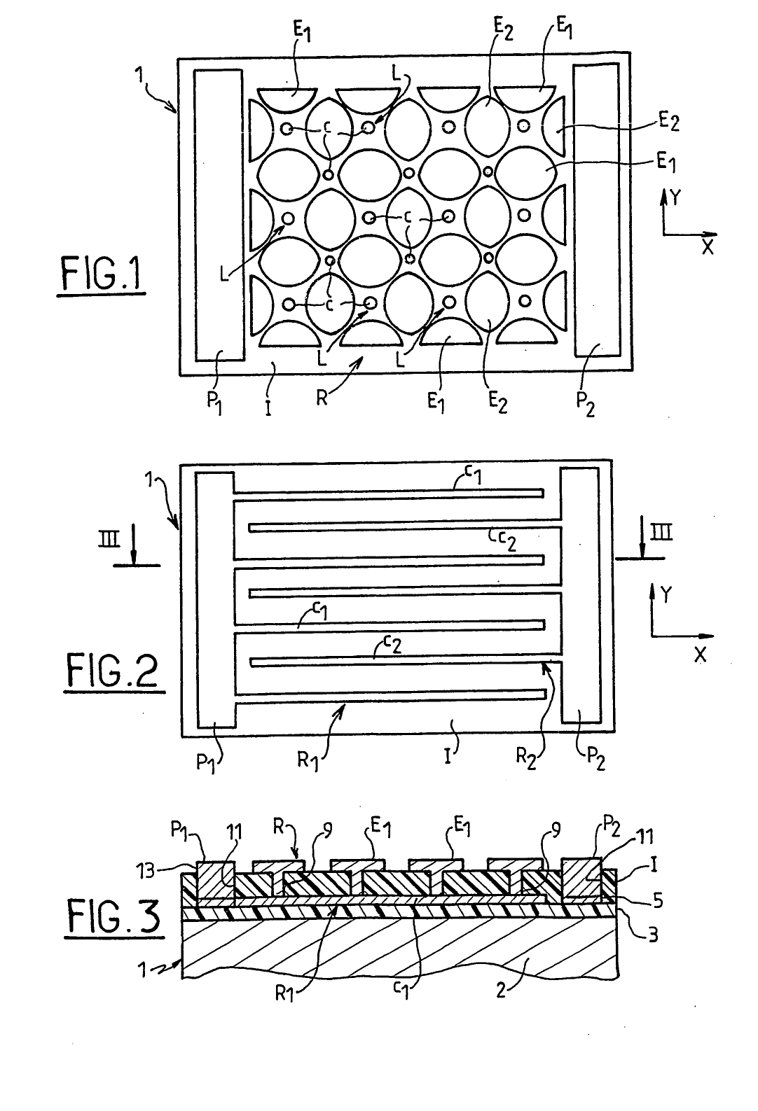

In the example shown in FIGS. 1 to 3, a regular array R of electrodes E₁ and E₂ is formed on the surface of the top insulating layer I of a multilayer substrate 1, and is connected to two power supply pads P₁ and P₂ via two arrays R₁ and R₂ of conductor tracks C₁ and C₂. The array R of electrodes E₁ and E₂ is designed to distribute an alternating electric field applied from the two power supply pads P₁ and P₂ in non-uniform manner and to define on the surface of the insulating layer I local zones L that are regularly spaced apart and in which the electric field is at a minimum.

In general, a local zone L is defined by an individual group of at least two pairs of electrodes, which corresponds to the example shown in FIG. 1. The electrodes E₁ and E₂ are regularly spaced apart in a plurality of rows parallel to an axis X, it being understood that the electrodes in any one row have the same polarity and that the electrodes in two adjacent rows are of opposite polarities. In other words, rows of electrodes E₂ are interposed between rows of electrodes E₁, or vice versa.

Each local zone L of minimum electrical field is thus defined between two adjacent electrodes E₁ or E₂ in a common row, and by two facing electrodes E₁ or E₂ located respectively in the two rows that are adjacent to said row. In this example, a given electrode E₁ or E₂ can thus be used to define four local zones L, and the electrodes of two adjacent rows are disposed in a staggered configuration.

FIGS. 2 and 3 show how the electrodes E₁ and E₂ are connected to the power supply pads P₁ and P₂′ This connection is provided by the two arrays R₁ and R₂ of parallel conductor tracks C₁ and C₂ likewise extending along the axis X. These two arrays R₁ and R₂ are connected respectively to the two power supply pads P₁ and P₂ on opposite sides of the array R of electrodes E₁ and E₂, the pads extending along an axis Y that is perpendicular to the axis X. Each power supply pad P₁ or P₂ together with its associated array R₁ or R₂ of conductor tracks forms a comb, and the two combs are interleaved in one another (FIG. 2). The two arrays R₁ and R₂ are received in the insulating layer I, i.e. the electrodes E₁ and E₂ are connected via a level that is different from that in which they are situated (FIG. 3), so that the principle whereby the electrodes are connected remains independent of the number of electrodes used.

An example of how the array R of electrodes and the connection arrays R₁ and R₂ to the power supply pads P₁ and P₂ can be fabricated is shown in FIG. 3, starting from a base substrate 2 constituted by a wafer of single crystal silicon that is lightly doped in order make the arrays R, R₁, and R₂.

In a first step, oxidation is used to form a layer of silicon oxide 3 which covers the surface of the substrate 1 over a thickness of about 50 nanometers (nm) so as to prevent electric field lines from passing via the substrate 1. In a second step, the layer 3 is covered in a conductive layer 5, e.g. of aluminum, which layer is deposited by evaporation to have a thickness of about 300 nm, and the arrays R₁ and R₂ of conductor tracks C₁ and C₂ together with the power supply pads P₁ and P₂ are formed therein by photolithography and by wet etching the aluminum. In a third step, the insulating layer I of silicon oxide deposited using the atmospheric pressure chemical vapor deposition (APCVD) technique or any other technique such as sputtering, for example, overlies the assembly, and by means of a mask and by photolithography together with plasma etching of the oxide layer using SF₆, small openings 9 that are regularly spaced apart along the conductor tracks C₁ and C₂ are made together with two large openings 11 in register with the power supply pads P₁ and P₂ In a fourth step, a new conductor layer 13 of aluminum is evaporated over the assembly to a thickness that is about 100 nm greater than that of the lower layer I, and the openings 9 and 11 are filled in so as to provide connections with the arrays R₁ and R₂ of conductor tracks C₁ and C₂. Finally, in a fifth step of photolithography and etching of the aluminum, the shape of the array R of electrodes E₁ and E₂ is determined.

In a variant of this embodiment, the base substrate 2 may be a plate of glass, and the array of electrodes R together with the arrays R₁ and R₂ can be made of material other than aluminum, e.g. of gold or chromium, by adapting the fabrication technique to the metal that is selected. What matters in the invention is that the array R of electrodes E₁ and E₂ and its connection to the power supply pads P₁ and P₂ via the two arrays R₁ and R₂ should be situated at different levels, i.e. that the substrate 1 should be of the multilayer type.

This characteristic makes it possible to fabricate a device having a high degree of integration. By way of non-limiting example, it is possible to fabricate a device having a side of 1 centimeter (cm) that carries 1000 to 50,000 local zones L. Although FIGS. 1 to 3 show only small numbers of electrodes E₁, E₂ and of local zones L, that is purely for reasons of clarity in the drawings.

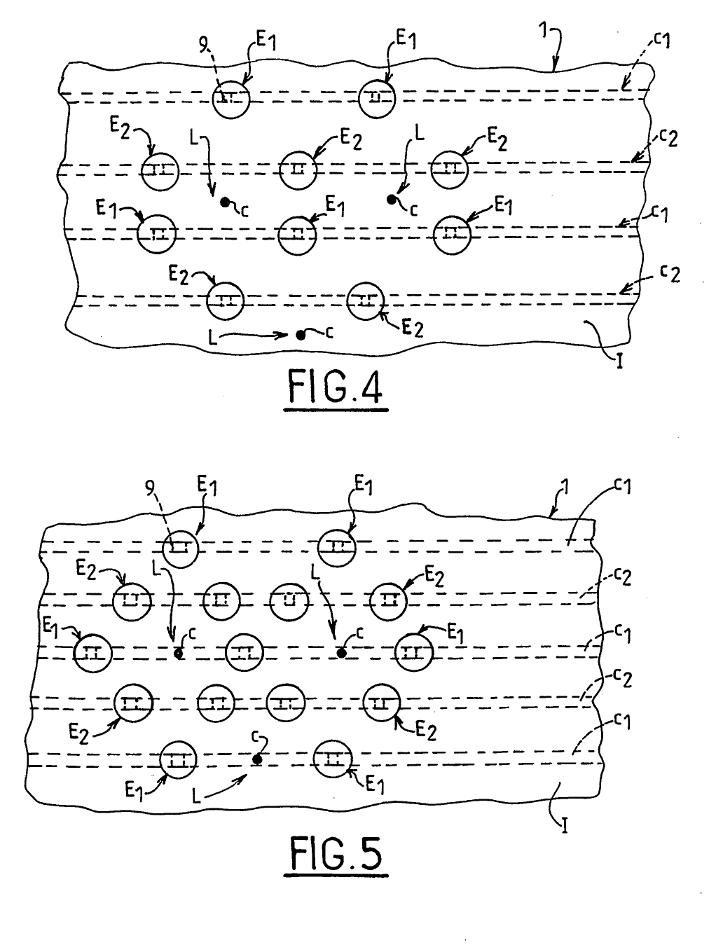

FIGS. 4 and 5 are diagrams showing two other possible forms for the array R of electrodes E₁ and E₂, it being understood that the electrodes E₁ and E₂ are connected to the power supply pads P₁ and P₂ via two arrays R₁ and R₂ of conductor tracks C₁ and C₂ in a manner similar to that shown in the example of FIG. 2. Each local zone L is defined by three pairs of electrodes E₁ and E₂ in FIG. 4, and by four pairs of electrodes E₁ and E₂ in FIG. 5. From these examples, it can be seen that a local zone L is defined from at least two pairs of electrodes E₁ and E₂, it being understood that the number of pairs of electrodes can itself be odd or even.

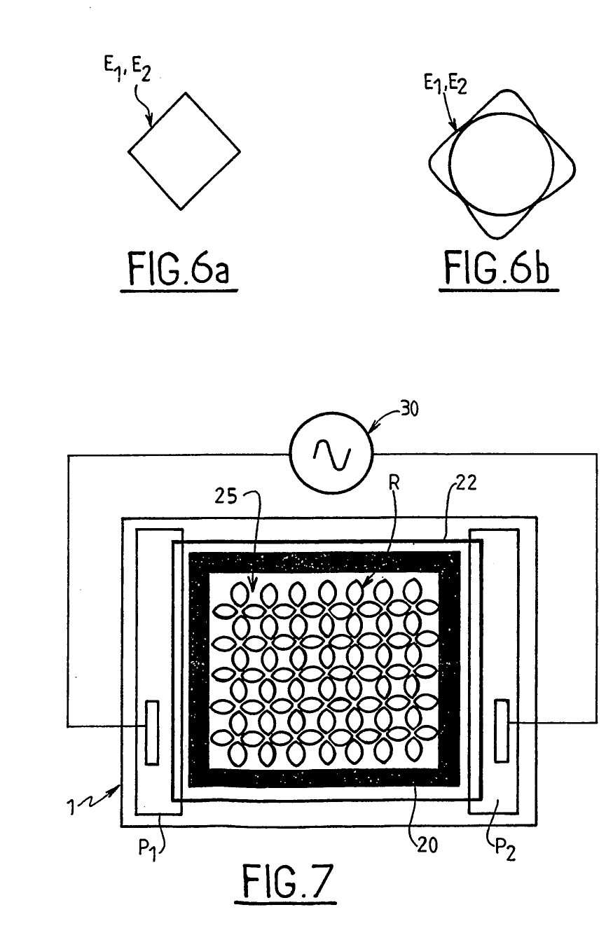

In the example of FIG. 1, the electrodes E₁ and E₂ are generally oval in shape or flower petal shape, and four electrodes define a local zone L by occupying a shape somewhat like a four-leaf clover, whereas the electrodes E₁ and E₂ are round in shape in the examples of FIGS. 4 and 5, it being understood that other shapes could be envisaged, for example they could be square in shape (FIG. 6a) or substantially square in shape (FIG. 6b), being symmetrical and having at least four corners (FIG. 6b), each corner of an electrode pointing towards the center of a local zone L.

An electric of a system for manipulating dielectric particles is shown diagrammatically in FIG. 7. The system comprises a substrate 1 as defined above together with its array R of electrodes E₁ and E₂, a sealing gasket 20 of silicone which surrounds the array R, and a glass plate 22 fitted onto the gasket 20 in order to define a chamber 25 that is to receive biological cells, e.g. in suspension in a medium and introduced into the chamber 25 by means of a pipette, for example. The two pads P₁ and P₂ are connected to an alternating voltage source 30. Naturally, the chamber 25 could be made in some other way.

In general, the system is more particularly designed for applying negative dielectrophoresis forces to the cells in suspension in the chamber 25.

To this end, action is taken on the frequency of the electric field, and electrical conductivity is selected that is appropriate to ensure that the medium is more polarizable than are the particles that are to be manipulated, thus making it possible to direct the particles towards the central portions of the local zones L by the action of negative dielectrophoresis forces, thereby concentrating them in a matrix array.

By acting on the electric field parameters, it is possible advantageously to direct the particles to the central points of the local zones L in such a manner as to encourage the particles to be concentrated in a manner that is regularly distributed over the surface of the insulating layer I of the substrate 1.

In order to show up this result, in FIG. 1 particle concentrations c are shown that are present in the central portions of the local zones L and that are regularly distributed over the surface of the substrate 1.

By way of example, the two pads P₁ and P₂ have been powered with a sinusoidal alternating voltage having an amplitude of about 5 volts (V) to 10 V peak-to-peak, and the frequency was caused to vary over a range of about 10 kilohertz (kHz) to 10 megahertz (MHz). In one particular example, for a medium having conductivity of 300 microsiemens per centimeter (μS·cm⁻¹), while using a frequency of about 100 kHz, and a sinusoidal voltage of about 5 V peak-to-peak, latex beads having a diameter of 3 micrometers (μm) were grouped together, it being understood that the electric field and medium conductivity parameters need to be adjusted as a function of the particle that is to be manipulated.

Once the cells have been distributed over the surface of the substrate, it is possible to proceed with electroporation or lysis thereof depending on the intended application. In general, the system of the invention can be used for performing high throughout screening of pharmacological substances, transferring genes in cells, . . . , and for separating two species of cell in solution, one species being oriented towards the centers of the local zones defined between the electrodes, while the other species is oriented towards the electrodes.

1. A system for using dielectrophoresis to manipulate dielectric particles, in particular biological cells in suspension in a medium and subjected to the action of an alternating electric field of distribution that is made to be non-uniform by means of a regular array of electrodes suitable for defining local zones where the electric field is at a minimum to concentrate particles in said local zones by the action of negative dielectrophoresis forces, wherein the array of electrodes is formed on the surface of a multilayer substrate, and wherein the same-polarity electrodes of the array are connected to respective common power supply pads via two arrays of conductor tracks which are formed at an intermediate level situated beneath the array of electrodes.

2. A system according to claim 1, wherein the multilayer substrate comprises at least one base substrate, a conductor layer being deposited on the base substrate to form the two arrays of conductor tracks, and an insulating layer deposited on the conductor layer for forming the array of electrodes.

3. A system according to claim 2, wherein the array of electrodes is connected to the arrays of conductor tracks via holes passing through the insulating layer.

4. A system according to claim 2, wherein the two arrays of conductor tracks are interdigitated.

5. A system according to claim 1, wherein the electrodes are regularly spaced apart in a plurality of rows parallel to an axis X, wherein the electrodes in any one row have the same polarity, and wherein the electrodes of two adjacent rows are of opposite polarities.

6. A system according to claim 1, wherein local zones for concentration particles are regularly spaced apart along a plurality of rows parallel to the axis X.

7. A system according to claim 1, wherein the electrodes are substantially circular in shape.

8. A system according to claim 1, wherein the electrodes are substantially square in shape having four corners, each corner of an electrode pointing towards the center of a local zone.

9. A system according to claim 1, wherein the electrodes are symmetrical in shape having at least four corners, each corner of an electrode pointing towards the center of a local zone.

10. A system according to claim 1, also including a chamber formed over the substrate to receive particles in suspension.

11. A system according to claim 10, wherein the chamber is defined by a sealing gasket which surrounds at least the array of electrodes, and by a plate fitted on the gasket, and wherein the system also includes an alternating voltage source for powering the two pads.

12. A system according to claim 1, wherein the multilayer substrate supports an array of electrodes suitable for defining a number of local zones of the order of 1000 to 50,000 for a support having a side of one centimeter.
